(19) United States
(12) Patent Application Publication
(10) Pub. No.: US 2023/0131926 A1
(43) Pub. Date: Apr. 27, 2023

(54) METHOD AND SYSTEM FOR DETECTING HEAVY METAL IONS

(71) Applicants: Hohai University Changzhou Campus, Changzhou (CN); Jiangsu Jimbio Technology Co., Ltd., Changzhou (CN); Changzhou Youchen Measurement and Control Technology Co., Ltd, Changzhou (CN); Kangdasi (Nanjing) Technology Co., Ltd, Nanjing (CN)

(72) Inventors: Xiaolu ZHU, Changzhou (CN); Hao CHENG, Changzhou (CN); Wenjie ZHAO, Changzhou (CN); Xudong WU, Changzhou (CN); Chunbo ZHU, Changzhou (CN)

(73) Assignees: Hohai University Changzhou Campus, Changzhou (CN); Jiangsu Jimbio Technology Co., Ltd., Changzhou (CN); Changzhou Youchen Measurement and Control Technology Co., Ltd, Changzhou (CN); Kangdasi (Nanjing) Technology Co., Ltd, Nanjing (CN)

(21) Appl. No.: 17/973,560

(22) Filed: Oct. 26, 2022

(30) Foreign Application Priority Data
Oct. 26, 2021 (CN) 202111250194.7

(51) Int. Cl. G01N 27/48; G01N 27/30

(52) U.S. Cl. CPC G01N 27/48; G01N 27/308; G01N 33/1813

(57) ABSTRACT

A method and a system for detecting heavy metal ions are provided. The method includes: fixing a pre-manufactured micro-electrode chip on a connecting device, connecting the connecting device to an electrochemical workstation, carrying out setting before detection on the electrochemical workstation, the micro-electrode chip including a counter electrode and a working electrode, carrying out graphene modification treatment on the counter electrode in advance, and carrying out bismuth film modification treatment on the working electrode in advance; and carrying out acid pretreatment on an aqueous solution to be detected, dropwise adding 5 µL to 100 µL of a treated aqueous solution to a working area of the pre-manufactured micro-electrode chip, measuring an I-V curve by the electrochemical workstation subjected to the setting before detection, and determining species and concentrations of heavy metal ions in the aqueous solution according to peak values of voltage and current of the I-V curve.

### CROSS REFERENCE TO THE RELATED APPLICATIONS

This application is based upon and claims priority to Chinese Patent Application No. 202111250194.7, filed on Oct. 26, 2021, the entire contents of which are incorporated herein by reference.

### TECHNICAL FIELD

The present disclosure belongs to the technical field of heavy metal ion detection, and particularly relates to a method and system for detecting heavy metal ions.

### BACKGROUND

Heavy metals are considered as a class of chemical substances having “low concentration components but high toxicity”, and also refers to a class of metals having density exceeding 4.5 g/cm3. These heavy metals are widely distributed in nature but difficult to biodegrade, thus causing great harm to the ecological environment. In the biological world, the heavy metals can produce enrichment effects by means of food chains, which will affect human life and health over time, and since it is easy for the heavy metals to bind to mercaptan groups in some proteins, the heavy metals will have an impact on normal life activities of organisms when entering the organisms, thereby producing toxicity. Some heavy metals such as Fe, Co, Zn and Mn are trace elements that are required for the organisms, and will produce toxic effects only at certain concentrations, and the heavy metals such as Hg, Pb and Cd are also considered to be toxic at low concentrations as elements that are not required for the organisms. In addition, excessive heavy metal ions can cause chromosomal diseases, and copper salts can destroy hemoglobin, causing cardiovascular and cerebrovascular diseases. Therefore, detection of heavy metal ions in aqueous solutions and test of biological toxicity have become an important topic of current research.

Presently, methods used for detecting heavy metal ions include: atomic absorption spectrometry (AAS), atomic fluorescence spectrometry (AFS), ultraviolet spectrophotometry (UV), X-ray fluorescence spectroscopy (XRF), inductively coupled plasma mass spectrometry (ICP-MS), and inductively coupled plasma (ICP). Although these methods play corresponding roles and are widely used, these detection methods still have corresponding defects, such as expensive cost, complex operations and weak sensitivity. With an increasing number of heavy metals and species in water, the harm to human beings is increasingly serious, and accordingly, it has become a problem to be urgently solved to find a simple and fast detection technology for heavy metal ions in water.

With limitations of a manufacturing process and use occasions, a traditional working electrode (WE) usually has a large external dimension reaching centimeters or above. A glassy carbon electrode (GCE) has excellent mechanical properties, the surface of which can be modified, and accordingly, is currently the most used electrode. However, the modified electrode sometimes can not be stored for a long time, and is vulnerable to various environmental factors during measurement, which is not conducive to on-site monitoring. Further, in order to achieve trace detection of target metal ions in micro samples, it is necessary to miniaturize a three-electrode system and modify the surface of the working electrode, which involves complex technical problems.

It is usually necessary to place a traditional reference electrode (RE) in a glass chamber filled with a protective liquid for protection, and to store the RE in a dark environment, and the electrode is likely to being damaged and has short life, and accordingly, cannot be used in miniaturization, high temperature and high pressure conditions. A traditional auxiliary (counter) electrode (CE) usually uses a platinum wire or a platinum sheet, produces the effect of forming a current loop with the WE, and basically does not participate in chemical reactions occurring in an electrolytic cell, and generally, it is only necessary for selection of the CE to satisfy requirements of excellent conductivity and stable electrochemical performance.

### SUMMARY

Aiming at the defects in the prior art, the objective of the present disclosure is to provide a method and system for detecting heavy metal ions. A miniaturized three-electrode system is used, such that species and concentrations of heavy metal ions may be detected at an extremely low concentration, detection may be carried out within a certain range, a detectable sample has a small size, operation is simple, selectivity on target metal ions is excellent, and detection sensitivity is high, such that the heavy metal ions may be qualitatively and quantitatively detected on line.

In order to solve the existing technical problem, the technical solution used by the present disclosure is as follows:
in a first aspect, the present disclosure provides a method for detecting heavy metal ions. The method includes:
fixing a pre-manufactured micro-electrode chip on a connecting device, connecting the connecting device to an electrochemical workstation, carrying out setting before detection on the electrochemical workstation, the micro-electrode chip including a counter electrode and a working electrode, carrying out graphene modification treatment on the counter electrode in advance, and carrying out bismuth film modification treatment on the working electrode in advance; and
carrying out acid pretreatment on an aqueous solution to be detected, dropwise adding 5 µL to 100 µL of an aqueous solution to be detected that is subjected to acid pretreatment, to a working area of the micro-electrode chip, measuring an I-V curve by means of the electrochemical workstation subjected to the setting before detection, and determining species and concentrations of heavy metal ions in the aqueous solution to be detected according to peak values of voltage and current of the I-V curve.

In combination with the first aspect, further, manufacturing the micro-electrode chip includes: when a micro-electrode is a planar electrode, sequentially carrying out oxidation, spin coating, exposure, development, metal deposition and photoresist removal treatment on a substrate, to obtain the micro-electrode chip; and when the micro-electrode is of a protruding structure, carrying out cleaning, thick photoresist coating, exposure, development, multi-angle surrounding metal deposition or sputtering and gap clearing treatment on the substrate.

In combination with the first aspect, further, the setting before detection includes: selecting “different pulse stripping voltammetry”, and setting detection parameters according to the species of the heavy metal ions to be detected, the setting before detection being used for detecting the I-V curve.

In combination with the first aspect, further, the carrying out acid pretreatment on an aqueous solution to be detected includes: dropwise adding a HAc—NaAc buffer solution to adjust a pondus hydrogenii (pH) value of the aqueous solution to be detected, and adjusting the pH value to 4.0.

In combination with the first aspect, further, the graphene modification treatment includes: dropwise adding 0.4 mg/mL graphene dispersion solution onto the counter electrode of the micro-electrode chip, and using cyclic voltammetry to scan 40 segments, to obtain a graphene-modified counter electrode.

In combination with the first aspect, further, during scanning, a scanning rate is 0.2 V/s, and a scanning range is -1.5 V to 0.6 V

In combination with the first aspect, further, the bismuth film modification treatment includes: dropwise adding 50 µL to 200 µL of 50 mg/L bismuth nitrate solution to the working area of the micro-electrode chip until the working area is completely covered, and selecting an “electro-deposition method” to plate bismuth for 240 s at a potential of -0.6 V to obtain a bismuth-film-modified working electrode.

In a second aspect, the present disclosure further provides a system for detecting heavy metal ions for executing the method for detecting heavy metal ions of any one of the first aspect. The system includes a micro-electrode chip, a connecting device, an electrochemical workstation and a computer, where a working electrode, a counter electrode and a reference electrode are arranged in a working area of the micro-electrode chip; the connecting device includes a clamping mechanism and a probe array, at least three probes in the probe array being arranged, the working electrode, the counter electrode and the reference electrode being arranged below three adjacent probes respectively and being fixed by means of clamping forces provided by the clamping mechanism, and upper portions of the probes being connected to the electrochemical workstation by means of wires; and the computer is connected to the electrochemical workstation and matches the electrochemical workstation to display a detection result.

In combination with the second aspect, further, the working electrode and the counter electrode are made of gold, and the reference electrode is made of a conductive silver adhesive or a gold layer structure covered with the conductive silver adhesive.

Compared with the prior art, the present disclosure has the beneficial effects:

according to the method and system for detecting heavy metal ions provided in the present disclosure, the pre-manufactured micro-electrode chip is fixed on the connecting device, the micro-electrode chip includes the counter electrode and the working electrode, graphene modification treatment is carried out on the counter electrode in advance, bismuth film modification treatment is carried out on the working electrode in advance, corrosion to the surface of the electrode may be reduced by means of graphene modification treatment of the electrode, the micro-electrode chip has stronger detection performance by means of bismuth film modification treatment of the working electrode, and moreover, retention time of a coating film is long, and a device may stably work for a long time. The connecting device is connected to the electrochemical workstation, setting before detection is carried out on the electrochemical workstation, the aqueous solution to be detected is pretreated, so as to obtain an optimal detection effect, 5 µL to 100 µL of the pretreated aqueous solution to be detected is dropwise added to the working area of the micro-electrode chip, and a detectable sample has a small size; the I-V curve is measured by means of the electrochemical workstation subjected to the setting before detection, the species and concentrations of the heavy metal ions in the aqueous solution to be detected are determined according to the peak values of voltage and current of the I-V curve, operation is simple, a detection limit is low, the species and concentrations of the heavy metal ions may be detected at the extremely low concentration, and detection may be carried out within a certain range.

According to the method and system for detecting heavy metal ions provided in the present disclosure, when the micro-electrode is of a protruding three-dimensional structure or structure array, contact area between the electrode and a liquid to be detected may be increased, thereby improving a lower limit and sensitivity of detection concentration; and a material is green and environment-friendly and has low cost, the defects that a traditional heavy metal detection instrument is complex, and has large sample demand, a narrow application field, high test cost, etc. are overcome, and the heavy metal ions may be qualitatively and quantitatively detected on line.

### BRIEF DESCRIPTION OF THE DRAWINGS

FIG. 1 is a schematic connection diagram of a system for detecting heavy metal ions provided in an example of the present disclosure;

FIGS. 2A-2B are schematic diagrams of a micro-electrode chip provided in an example of the present disclosure;

FIGS. 3A-3C are test result graphs provided in an example of the present disclosure;

FIG. 4 is a schematic structural diagram of a connecting device provided in an example of the present disclosure; and

FIG. 5 is a flow diagram of a method for detecting heavy metal ions provided in an example of the present disclosure.

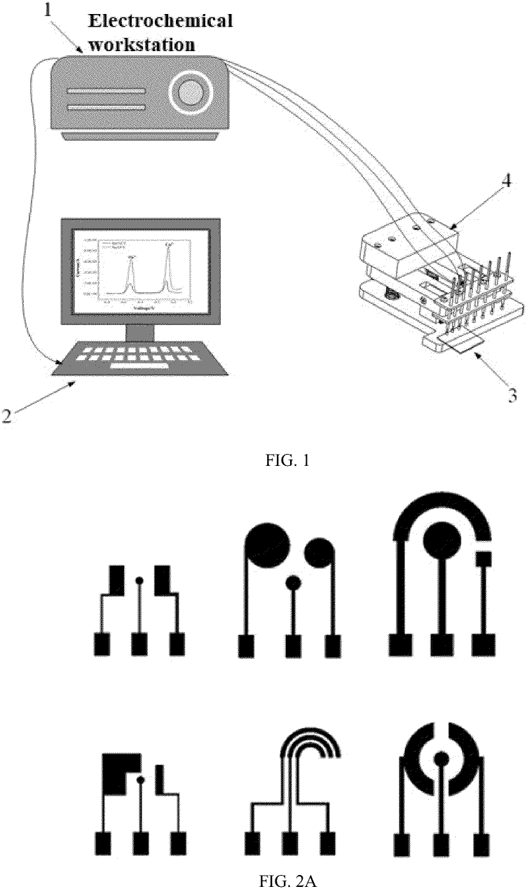

In the figures: 1, electrochemical workstation; 2, computer; 3, micro-electrode chip; 31, counter electrode; 32, reference electrode; 33, working electrode; 34, wiring terminal; 4, connecting device; 41, handle; 42, return plate; 43, return spring; 44, base; 45, supporting column; 46, probe array; 47, fixing plate; 48, adjustment mechanism; and 49, binding post.

### DETAILED DESCRIPTION OF THE EMBODIMENTS

The present disclosure is further described below. The following examples are merely used for illustrating the technical solution of the present disclosure more clearly, and may not limit the scope of protection of the present disclosure.

### Example 1

As shown in FIG. 5, a method for detecting heavy metal ions provided in an example of the present disclosure included:

S1, fix a pre-manufactured micro-electrode chip on a connecting device, connect the connecting device to an electrochemical workstation, carry out setting before detection on the electrochemical workstation, the micro-electrode chip including a counter electrode and a working electrode, carry out graphene modification treatment on the counter electrode in advance, and carry out bismuth film modification treatment on the working electrode in advance.

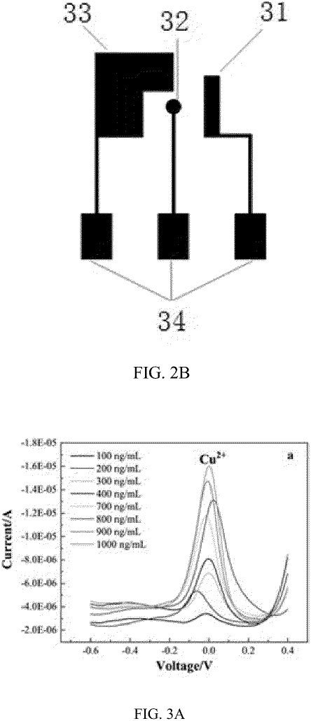

The micro-electrode chip was pre-manufactured before detection, the micro-electrode chip shown in FIGS. 2A-2B was manufactured by using computer-aided design (CAD) software design and a photoetching process, and stripping volt-ampere curves of different shapes of electrodes for detecting target heavy metal ions may be different, such that ideal stripping peak current was obtained. Redox reaction of the heavy metal ions occurred on the working electrode, such that the working electrode was designed into a square shape, a circular shape and a semi-annular shape as shown in FIG. 2A, the working electrode and the counter electrode were made of gold, the reference electrode was made of 3701 conductive silver adhesive, and the electrode transformed by the 3701 conductive silver adhesive was used as a reference electrode and was used for comparing and computing a potential of the working electrode as a reference in a detection process. The working electrode may also be referred to as WE, the counter electrode may also be referred to as CE, and the reference electrode may also be referred to as RE.

The micro-electrode chip was processed by means of a photoetching technology, the photoetching technology was widely used for fields of microelectronics, etc., and simply, patterns on a mask were transferred to a substrate by means of a light source. When a micro-electrode was a planar electrode, oxidation, spin coating, exposure, development, metal deposition and photoresist removal treatment were sequentially carried out on the substrate, to obtain the micro-electrode chip. When the micro-electrode was of a protruding structure, cleaning, thick photoresist coating, exposure, development, multi-angle surrounding metal deposition or sputtering and gap clearing treatment were carried out on the substrate. By means of gap clearing, gaps between the three-dimensional electrodes may be constructed by means of a secondary etching method, and gaps may also be etched at proper positions covered with metal layers by means of a mechanical cutter. The micro-electrode chip was obtained, the most important material of the photoetching technology was a photoresist, and pattern manufacturing may be completed by utilizing optical characteristics of the photoresist.

After the micro-electrode chip was manufactured by means of the photoetching technology, it was necessary to carry out counter electrode graphene modification treatment on the counter electrode of the micro-electrode chip and working electrode bismuth film modification treatment on the working electrode of the micro-electrode chip. The counter electrode graphene modification treatment specifically included: dropwise add 0.4 mg/mL graphene dispersion solution onto the counter electrode of the micro-electrode chip, and use cyclic voltammetry to scan 40 segments at a scanning rate of 0.2 V/s in a range of -1.5 V to 0.6 V, to obtain a graphene-modified counter electrode, so as to reduce corrosion to a surface of the counter electrode. The working electrode bismuth film modification treatment specifically included: plate a bismuth film at a position of the working electrode by using the same bismuth film plating method, dropwise add 50 µL to 200 µL of a bismuth nitrate solution having a concentration of 50 mg/L to the working area of the micro-electrode chip until the working area is completely covered, connect the electrochemical workstation, and select an electrodeposition method (i-t curve) to plate bismuth for 240 s at a potential of -0.6 V to obtain a bismuth-film-modified working electrode. Therefore, the micro-electrode chip had stronger detection performance. If the working electrode was formed by depositing gold on a surface of the protruding structure or a micro-array structure (such as a square column, a cylinder, a polygon prism and an irregular shape), a volume of the bismuth nitrate solution may be properly increased when a bismuth film is plated, such that the protruding three-dimensional micro-electrode structure was completely covered with and immersed by the bismuth nitrate solution; and films may be intermittently plated repeatedly, and a liquid may be intermittently and properly shaken, so as to ensure and enhance integrity and a full-surface coverage degree of the plated bismuth film.

The micro-electrode chip was fixed on the connecting device, the connecting device was in communication with the electrochemical workstation, and in this case, setting before detection started to be carried out on the computer in communication with the electrochemical workstation and specifically included: select “different pulse stripping voltammetry”, and setting detection parameters according to the species of the heavy metal ions to be detected. The setting before detection was used for detecting the I-V curve (volt-ampere characteristic curve).

S2, carry out acid pretreatment on an aqueous solution to be detected, dropwise add 5 µL to 100 µL of an aqueous solution to be detected that is subjected to acid pretreatment, to a working area of the micro-electrode chip, measure an I-V curve by means of the electrochemical workstation subjected to the setting before detection, and determine species and concentrations of heavy metal ions in the aqueous solution to be detected according to peak values of voltage and current of the I-V curve.

Before the species and concentrations of the heavy metal ions in the aqueous solution to be detected were detected, pondus hydrogenii (pH) value pretreatment was carried out on the aqueous solution to be detected. The pH value pretreatment specifically included: use a titration method to adjust a pH value of the aqueous solution to be detected, dropwise add a HAc—NaAc buffer solution to adjust the pH value of the aqueous solution to be detected, and adjust the pH value to 4.0, so as to obtain an optimal detection effect.

5 µL of the pretreated aqueous solution to be detected was dropwise added to the working area of the micro-electrode chip, to ensure that all the electrodes in the working area were in communication by the aqueous solution to be detected, the parameters in S1 were kept unchanged, and the I-V curve was tested, and the species and concentrations of the heavy metal ions in the aqueous solution to be detected may be determined according to peak values of voltage and current of the I-V curve (volt-ampere characteristic curve) measured by the electrochemical workstation.

The measured I-V curve (volt-ampere characteristic curve) was compared with each group of curves measured under a standard concentration condition, the species and concentrations of the measured trace heavy metal ions were analyzed by means of a function method, the concentrations of the metal ions in different aqueous solutions may be determined according to a relative current value (peak current-base current), and a water content of the measured aqueous solution was determined according to the voltage corresponding to the peak current of the measured I-V curve (volt-ampere characteristic curve).

### Example 2

An example of the present disclosure provided a method for detecting heavy metal ions. A micro-electrode chip shown in FIGS. 2A-2B was selected to construct a standard three-electrode system, two gold electrodes were used as a working electrode and a counter electrode, an electrode transformed by 3701 conductive silver adhesive was used as a reference electrode, the micro-electrode chip was connected to an electrochemical workstation by means of a matched connecting device, and a computer in communication with the electrochemical workstation was started.

100 µL of the pretreated aqueous solution to be detected as described in example 1 was dropwise added right above the micro-electrode chip during detection, to ensure that a liquid completely covers three electrodes, an acetic acid-sodium acetate solution having a pH value of 4.0 was selected as a buffer solution, and a certain amount of copper ion standard solution was sequentially added, such that a gradient concentration of copper ions contained in the buffer solution was 100 ng/mL, 200 ng/mL, 300 ng/mL, 400 ng/mL, 700 ng/mL, 800 ng/mL, 900 ng/mL and 1000 ng/mL, differential pulse stripping voltammetry was selected, an enrichment potential was configured to be -0.6 V, enrichment time was configured to be 60 s, and a scanning potential was configured to be -0.6 V to 0.4 V to explore an influence of a change of a copper ion concentration on stripping peak current.

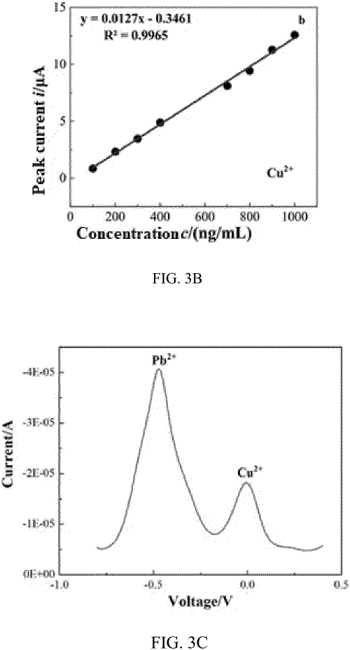

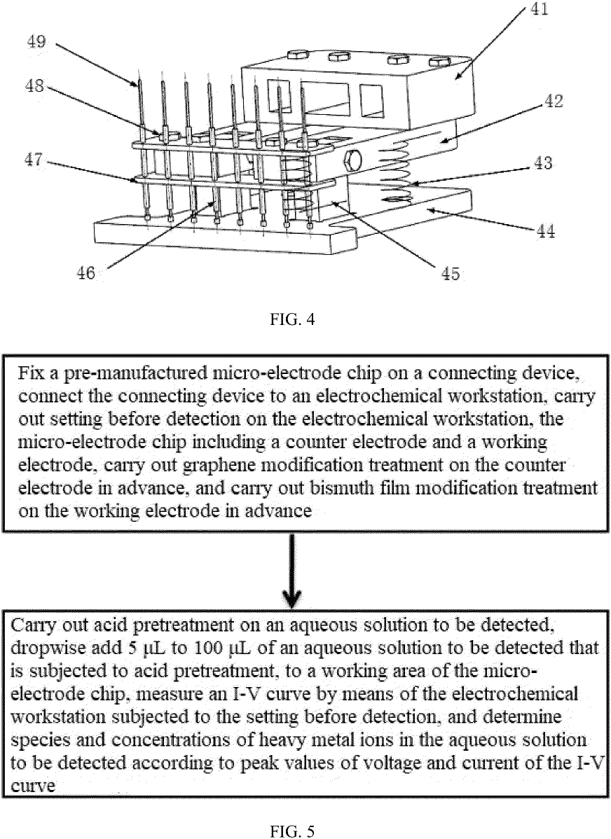

FIG. 3A was a differential pulse stripping voltammetry graph for copper ion detection. FIG. 3B was a graph of a linear relation between peak current intensity and a copper ion concentration. It may be seen from figures that copper ions showed a stripping peak around 0 V, the stripping peak current intensity was linearly related to copper ion concentration in a concentration range of 100 ng/mL to 1000 ng/mL, a linear equation was iₚ(Cu2+)(µA)=0.0127C_{Cu(II)}(ng/mL)-0.3461, and a correlation coefficient was R2=0.9965.

The differential pulse stripping voltammetry was used, the square micro-electrode chip was utilized to match the connecting device to quantitatively detect the copper ions in water, the stripping peak obtained by detecting the copper ions was clear and sharp, and compared with a traditional three-electrode, the sample amount required for the method was about 100 µL, and was reduced by 100 times or above, no stirring device was required in an experiment process, and measurement time was only 1 min, thereby effectively reducing time cost.

FIG. 3C showed that simultaneous detection of copper ions and lead ions in an acetate buffer liquid. It may be seen that corresponding stripping peaks of the lead ions and the copper ions occurred around -0.5 V and 0 V respectively, which did not interfere with each other, the copper ions and the lead ions may be simultaneously detected, and in addition, the method may be used for subsequent cell toxicity experiments.

### Example 3

The present disclosure further provided a system for detecting heavy metal ions for executing the method for detecting heavy metal ions of example 1. The system included a micro-electrode chip, a connecting device and an electrochemical workstation.

As shown in FIG. 2B, a working electrode, a counter electrode, a reference electrode and a wiring terminal were arranged in a working area of the micro-electrode chip.

The connecting device included a clamping mechanism and a probe array. The probe array included at least three probes, where a top end of each of the probes was provided with a binding post, and the probes may be connected to the electrochemical workstation by means of wires. The clamping mechanism included a handle, a return plate, a return spring, a base, a supporting column, fixing plates and an adjustment mechanism, where a circular concave hole for accommodating a bottom end of the return spring was provided on the base, the return plate was arranged at a top end of the return spring, the handle was arranged above the return plate, the handle pressed the return spring and was fixedly connected to the return plate, the supporting column was further arranged on the base, a bottom end of the supporting column was fixedly connected to the base, a top end of the supporting column was rotatably connected to the return plate, and clamping forces may be provided by means of the base, the supporting column, the return plate, the return spring and the handle.

The fixing plates were fixedly connected to an upper portion and a lower portion of one end of the return plate away from the return spring, through holes for the probes to pass therethrough were provided on the fixing plates, the probe array was arranged in the through holes and fixed by the fixing plates, the micro-electrode chip may be fixed on the connecting device in cooperation with the clamping forces, and may be connected to the electrochemical workstation in cooperation with the wires.

Upper portions of the probes were connected to the electrochemical workstation by means of the wires, and a computer was connected to the electrochemical workstation and cooperated with the electrochemical workstation to display a detection result.

The pre-manufactured micro-electrode chip was fixed on the connecting device, the counter electrode and the working electrode of the micro-electrode chip were treated, the connecting device was connected to the electrochemical workstation, and setting before detection was carried out on the electrochemical workstation; the aqueous solution to be detected was pretreated, 5 µL to 100 µL of the pretreated aqueous solution to be detected was dropwise added to the working area of the micro-electrode chip, to measure the I-V curve (volt-ampere characteristic curve), and the species and concentrations of the heavy metal ions in the aqueous solution to be detected were determined according to the peak values of voltage and current of the I-V curve (volt-ampere characteristic curve).

What is described above is merely the preferred implementation of the present disclosure, it should be pointed out that those of ordinary skill in the art may further make some improvements and deformations without departing from the principle of the present disclosure, and these improvements and deformations should also fall within the scope of protection of the present disclosure.

What is claimed is:

1. A method for detecting heavy metal ions, comprising:
fixing a pre-manufactured micro-electrode chip on a connecting device, connecting the connecting device to an electrochemical workstation, carrying out a setting before a detection on the electrochemical workstation, the pre-manufactured micro-electrode chip comprising a counter electrode and a working electrode, carrying out a graphene modification treatment on the counter electrode in advance, and carrying out a bismuth film modification treatment on the working electrode in advance; and
carrying out an acid pretreatment on an aqueous solution to be detected to obtain a treated aqueous solution, dropwise adding 5 µL to 100 µL of the treated aqueous solution to a working area of the pre-manufactured micro-electrode chip, measuring an I-V curve by the electrochemical workstation subjected to the setting before the detection, and determining species and concentrations of the heavy metal ions in the aqueous solution to be detected according to peak values of a voltage and a current of the I-V curve.

2. The method for detecting the heavy metal ions according to claim 1, wherein manufacturing the pre-manufactured micro-electrode chip comprises: when a micro-electrode is a planar electrode, sequentially carrying out an oxidation, a spin coating, an exposure, a development, a metal deposition, and a photoresist removal treatment on a substrate to obtain the pre-manufactured micro-electrode chip; and when the micro-electrode is of a protruding structure, carrying out a cleaning, a thick photoresist coating, the exposure, the development, a multi-angle surrounding metal deposition, or a sputtering and gap clearing treatment on the substrate.

3. The method for detecting the heavy metal ions according to claim 1, wherein the setting before the detection comprises: selecting a different pulse stripping voltammetry, and setting detection parameters according to the species of the heavy metal ions to be detected, the setting before the detection being used for detecting the I-V curve.

4. The method for detecting the heavy metal ions according to claim 1, wherein the carrying out the acid pretreatment on the aqueous solution to be detected comprises: dropwise adding a HAc—NaAc buffer solution to adjust a pondus hydrogenii (pH) value of the aqueous solution to be detected to 4.0.

5. The method for detecting the heavy metal ions according to claim 1, wherein the graphene modification treatment comprises: dropwise adding a 0.4 mg/mL graphene dispersion solution onto the counter electrode of the pre-manufactured micro-electrode chip, and using a cyclic voltammetry to scan 40 segments to obtain a graphene-modified counter electrode.

6. The method for detecting the heavy metal ions according to claim 5, wherein during a scanning, a scanning rate is 0.2 V/s, and a scanning range is -1.5 V to 0.6 V.

7. The method for detecting the heavy metal ions according to claim 1, wherein the bismuth film modification treatment comprises: dropwise adding 50 µL to 200 µL of a 50 mg/L bismuth nitrate solution to the working area of the pre-manufactured micro-electrode chip until the working area is completely covered, and selecting an an electro-deposition method to plate bismuth for 240 s at a potential of -0.6 V to obtain a bismuth-film-modified working electrode.

8. A system for detecting heavy metal ions for executing the method for detecting the heavy metal ions according to claim 1, comprising the pre-manufactured micro-electrode chip, the connecting device, the electrochemical workstation, and a computer, wherein the working electrode, the counter electrode, and a reference electrode are arranged in the working area of the pre-manufactured micro-electrode chip; the connecting device comprises a clamping mechanism and a probe array, at least three probes in the probe array are arranged, the working electrode, the counter electrode, and the reference electrode are arranged below three adjacent probes respectively and fixed by clamping forces provided by the clamping mechanism, and upper portions of the at least three probes are connected to the electrochemical workstation by wires; and the computer is connected to the electrochemical workstation and matches the electrochemical workstation to display a detection result.

9. The system for detecting the heavy metal ions according to claim 8, wherein the working electrode and the counter electrode are made of gold, and the reference electrode is made of a conductive silver adhesive or a gold layer structure covered with the conductive silver adhesive.

10. The system for detecting the heavy metal ions according to claim 8, wherein the pre-manufactured micro-electrode chip is manufactured as follows: when a micro-electrode is a planar electrode, sequentially carrying out an oxidation, a spin coating, an exposure, a development, a metal deposition, and a photoresist removal treatment on a substrate to obtain the pre-manufactured micro-electrode chip; and when the micro-electrode is of a protruding structure, carrying out a cleaning, a thick photoresist coating, the exposure, the development, a multi-angle surrounding metal deposition, or a sputtering and gap clearing treatment on the substrate.

11. The system for detecting the heavy metal ions according to claim 8, wherein in the method for detecting the heavy metal ions, the setting before the detection comprises: selecting a different pulse stripping voltammetry, and setting detection parameters according to the species of the heavy metal ions to be detected, the setting before the detection being used for detecting the I-V curve.

12. The system for detecting the heavy metal ions according to claim 8, wherein in the method for detecting the heavy metal ions, the carrying out the acid pretreatment on the aqueous solution to be detected comprises: dropwise adding a HAc—NaAc buffer solution to adjust a pondus hydrogenii (pH) value of the aqueous solution to be detected to 4.0.

13. The system for detecting the heavy metal ions according to claim 8, wherein in the method for detecting the heavy metal ions, the graphene modification treatment comprises: dropwise adding a 0.4 mg/mL graphene dispersion solution onto the counter electrode of the pre-manufactured micro-electrode chip, and using a cyclic voltammetry to scan 40 segments to obtain a graphene-modified counter electrode.

14. The system for detecting the heavy metal ions according to claim 13, wherein during a scanning, a scanning rate is 0.2 V/s, and a scanning range is -1.5 V to 0.6 V.

15. The system for detecting the heavy metal ions according to claim 8, wherein in the method for detecting the heavy metal ions, the bismuth film modification treatment comprises: dropwise adding 50 µL to 200 µL of a 50 mg/L bismuth nitrate solution to the working area of the pre-manufactured micro-electrode chip until the working area is completely covered, and selecting an an electro-deposition method to plate bismuth for 240 s at a potential of -0.6 V to obtain a bismuth-film-modified working electrode.
